(19) United States
(12) Patent Application Publication
(10) Pub. No.: US 2008/0082056 A1
(43) Pub. Date: Apr. 3, 2008

(54) INTRODUCER ASSEMBLY AND METHOD THEREFOR

(75) Inventors: Grant A. Mauch, Delano, MN (US); Brian Anderson, Buffalo, MN (US); Kevin Pietsch, Greenfield, MN (US); Steve Moreland, Aitkin, MN (US)

Correspondence Address: SCHWEGMAN, LUNDBERG & WOESSNER, P.A., P.O. BOX 2938, MINNEAPOLIS, MN, 55402, US

(73) Assignee: ENPATH MEDICAL INC., PLYMOUTH, MN (US)

(21) Appl. No.: 11/537,919

(22) Filed: Oct. 2, 2006

(51) Int. Cl. A61M 5/00

(52) U.S. Cl. 604/263

(57) ABSTRACT

An introducer assembly includes a sheath having a sheath proximal end and distal end, and a passage therethrough. The introducer assembly further includes a handle assembly that is mechanically and/or bonded coupled with a tubular sheath.

### TECHNICAL FIELD

Introducers and introducing assemblies, and more specifically an introducer assembly including a bonded sheath assembly.

### BACKGROUND

Introducer devices provide for access to the vascular system and are employed for inserting medical devices such as catheters, guidewires, leads, infusion ports, dialysis ports, dialysis catheters, and others. A typical procedure for gaining access to the central venous system or the arterial system with an introducer is the Seldinger Introduction Method. The Seldinger Method provides for insertion of a needle into the vasculature of a patient. Once the needle is in the vessel, the physician aspirates the needle to assure that the needle is in the vessel, and to draw out air present in the bore of the needle. The syringe is removed and discarded. A guide wire is inserted through the needle, and the needle is removed over the guide wire. The introducer, which includes a dilator and the sheath, is placed over the guidewire and inserted into the vessel. With the introducer and wire guide in the vessel, the dilator and wire guide are removed leaving only the sheath in the vessel. The desired medical device is implanted through the passage of the sheath.

The sheath is optionally removed from the medical device. Some removable sheaths are formed of slippery material, which is difficult to effectively couple or seal with other components. Furthermore, the introducer device provides access to the vein or artery, and therefore control of bleeding and the intake of air is necessary, for example, through use of a valve.

Accordingly, what is needed is an introducer assembly which can effectively seal against a wide variety of instruments without inhibiting the throughput of the instrument, or damaging the instrument. What is also needed is an introducer assembly which does not distract or interfere with the implantation process.

### BRIEF DESCRIPTION OF THE DRAWINGS

FIG. 1A illustrates a perspective view of an introducing assembly as constructed in accordance with at least one embodiment;

FIG. 1B illustrates a cross-sectional view of a portion of an introducing assembly as constructed in accordance with at least one embodiment;

FIG. 2 illustrates side view of a portion of a sheath assembly as constructed in accordance with at least one embodiment;

FIG. 3 illustrates side view of a portion of a sheath as constructed in accordance with at least one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

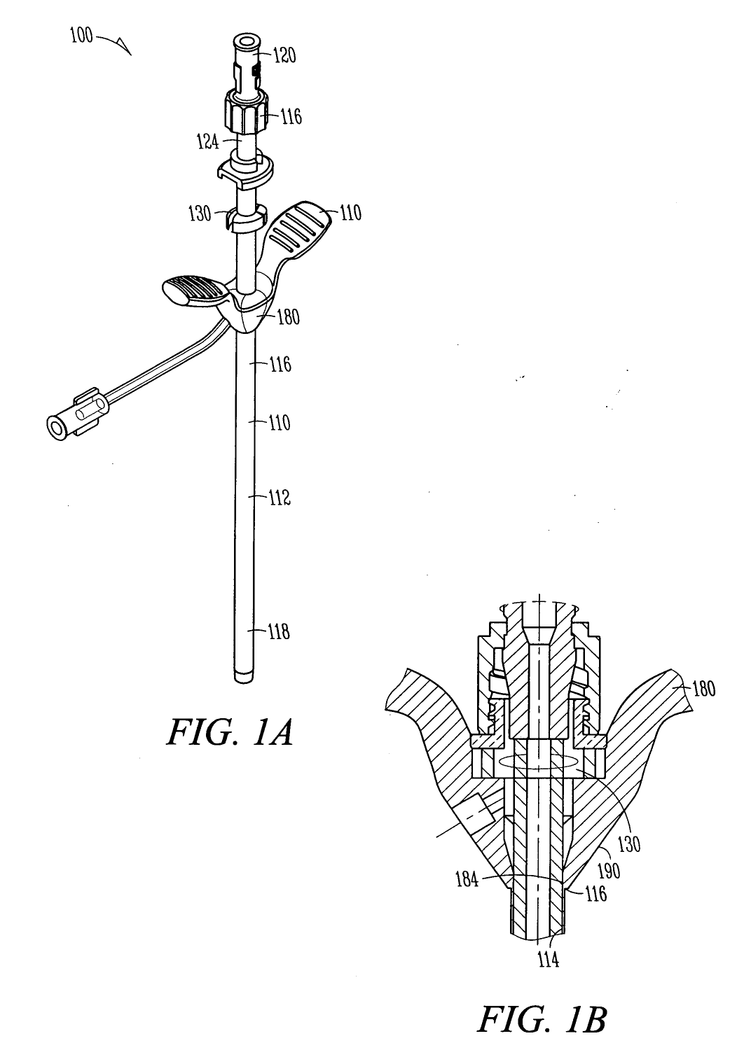

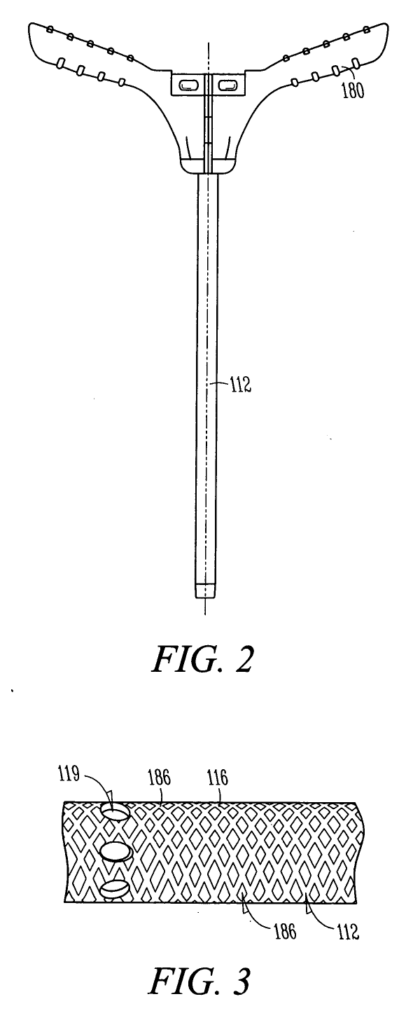

An introducer assembly 100 is illustrated in FIGS. 1A and 1B. The introducer assembly includes a sheath assembly 110 having a sheath 112 with a passage 114 therethrough. The sheath 112 is coupled with a handle assembly 180 as further described below. The sheath 112 extends from a sheath proximal end portion 116 to a sheath distal end portion 118, and is defined in part by a longitudinal axis. Near the sheath distal end portion 118 is a tapered portion, allowing for a more tapered transition portion to taper to the dilator disposed therethrough.

The sheath 112 is formed of, in an example, fluorinated polymers such as, but not limited to, PTFE (PolyTetraFluoroEthylene), FEP (Fluorinated Ethylene-Propylene), or polyimide. These materials assist in provided lubricious surface proprieties. The sheath material, such as the PTFE, can be molecularly oriented for optionally splitting the sheath. The molecularly oriented sheaths do not necessarily require an additional mechanical scoring operation to produce split lines. Instead, the oriented molecules allow the sheath 112 to naturally peel like a banana.

In a further option, the sheath 112 includes various types of sheaths, for instance, the sheath 112 can comprise a sheath which has a strengthening material, such as a strengthening braid of material. Alternatively, the sheath 112 includes a sheath which is modified to assist in preventing bends and/or kinks along the sheath.

The introducer assembly 100 further includes an instrument such as a dilator 120 that can be coupled with the sheath assembly 110, for example, with a rotatable coupler 116. For example, the rotatable coupler 116 includes a threaded portion that engages a projection or thread on the sheath assembly 110. The dilator 120 is removably disposed within a passage 114 of the sheath 112, and optionally is coaxial with the sheath 112. The sheath 112 includes a support diameter which is sized to receive a dilator 120 having a dilator diameter therethrough. It should be noted that other instruments such as leads and/or guidewires can be disposed through the sheath and sheath passage 114, as will further be described below. The dilator 120 extends from a dilator distal end to a dilator proximal end 124, where the dilator distal end is insertable into a patient, for example, over a needle or a guidewire. The dilator distal end optionally ends in a tapered end, allowing for ease of transition within tissue of a patient. The dilator proximal end 124 optionally includes features, such as a luer hub or threads, that allows for other devices to be coupled thereto.

In one embodiment, the handle assembly 180 and the sheath 112 are removable from around instruments disposed therein, such as a lead disposed with the sheath 112. For example, the sheath 112 is removable from around the instrument without having to slide or otherwise manipulate the introducer and/or the sheath over a proximal end of the instrument. In one option, the handle assembly 180 and/or the sheath 112 are removed from an outer perimeter along a cross-section of an instrument disposed therethrough.

The sheath 112 and/or the handle assembly 180, for example, can be removed from the instrument disposed therethrough in a number of different manners. For example, the sheath 112 can include structure integral therewith or non-integral that allows for the sheath 112 to be separated from around the instrument without damaging the instrument, and/or allows for the sheath 112 to be removed from the outer perimeter of the cross-section of the instrument. In some examples, the sheath 112 is coupled with a handle assembly 180, and the handle assembly 180 includes one or more tabs that are connected with the sheath 112 to tear the sheath 112 off of the instrument. In another example, the structure includes a tear strip, molecularly orientated material within the sheath, one or more openings in the sheath 112 allowing the sheath 112 to separate at one or more locations that each can be used alone or in combination to separate the sheath 112 from around the instrument. In another option, the sheath 112 is at least partially dissolvable within a body, allowing the sheath 112 to be removed from the instrument. In another option, a slitting or splitting device such as a slitter can be used to removed the sheath 112, where the sheath 112 is removed by slitting. In yet another option, the sheath further includes one, two or more tabs which can be used to separate the sheath away from the instrument. Further options include a pre-weakened or scored sheath, allowing for the sheath to be manually removed by tearing, separating, or slitting, for example. In yet another example, the sheath includes molecularly oriented material allowing for the sheath 112 to be removed from around the instrument.

The introducer assembly 100 optionally includes a valve 130 that is sealingly associated with the passage 114 of the sheath 112, allowing for substantial sealing of the passage 114. The valve 130 assists in preventing fluids to exit from a patient when the sheath 112 is disposed within the patient. The valve 130 assists in preventing fluids from exiting, yet permits passage of instruments through the valve 130, and in an option, substantially seals against the instruments that are disposed therethrough.

The valve 130 is coupled with a portion of the introducer 110, for example, within the handle assembly 180 of the introducer. The valve 130, in an option, is removable from around an outer cross-sectional perimeter of an instrument disposed through the introducer. For example, the valve 130 can include a mechanical weakening allowing for the valve 130 to slide off to the side of the instrument. Alternatively, the mechanical weakening can allow for the valve 130 to be torn or split away from the introducer. In yet another option, the valve 130 forms an adaptor that is attachable and removable by the user before, during, or after an implant procedure. For example, the user can remove or attach the valve assembly 130 with a fitting or other coupling.

As mentioned above, the handle assembly 180 is coupled to the sheath 112, where they are coupled together at an interface 190. In an option, the interface 190 includes a proximal end portion 116 of the sheath 112 and/or a portion of the handle assembly 180, such as in inner diameter 184. In an option, the interface 190, such as the sheath proximal end portion 116 and/or the inner or outer diameter of the handle assembly 180 includes a textured portion 186, such as shown in FIG. 3. In an option, the textured portion extends around an outer circumference of the sheath 112.

The textured portion 186 is formed in an option by chemically etching, for example, the sheath proximal end portion 116. In an example, the sheath 112 is rinsed with a solution, such as alcohol. The sheath 112 and/or the handle assembly 180 are chemically etched with a solution such as, but not limited to sodium naphthalene/ethylene glycol dimethyl ether solution.

The handle assembly 180 is coupled to the sheath 112, in an example, by overmolding the handle assembly 180 over the sheath 112. In another option, the handle assembly 180 can be preformed, and coupled with the sheath 112 by applying energy to the handle assembly 180 and/or the sheath 112, such as applying heat. During the process, the material of the handle assembly 180 bonds with the sheath 112, and chemically bonds with the chemically etched portion. In a further option, one or more flow holes 1 19 are formed in the sheath 112, such as by punching, prior to coupling the handle assembly 180 thereto. The flow holes 119 allow for material of the handle assembly 180 to flow therethrough, and further permit a mechanical bond of the handle assembly 180 and the sheath 112.

Advantageously, the introducer assembly described above provides many benefits. For example, the introducer assembly allows for a sheath, such as a slippery sheath, to be effectively bonded with a handle assembly, and further provides a seal between the sheath and the handle. For example, a seal is provided when the sheath is chemically bonded with the handle assembly. Furthermore, the methods and coupling techniques increase the tensile strength of the sheath to handle the bonding of the sheath and the handle assembly. In addition, the chemically etched sheath can withstand higher temperatures, for example temperatures in certain manufacturing procedures, such as, but not limited to during overmolding processes. The introducer assembly further allows for removal of the introducer without disruption to the procedure or placement of the medical device such as a lead.

It is to be understood that the above description is intended to be illustrative, and not restrictive. Many other embodiments will be apparent to those of skill in the art upon reading and understanding the above description. It should be noted that embodiments or portions thereof discussed in different portions of the description or referred to in different drawings can be combined to form additional embodiments of the present invention. The scope of the invention should, therefore, be determined with reference to the appended claims, along with the full scope of equivalents to which such claims are entitled.

What is claimed is:

1. An introducer assembly comprising:
a sheath extending from a sheath proximal end to a sheath distal end, the sheath having a passage therethrough;
a handle assembly coupled with the sheath at an interface; and
the interface having a textured portion at least prior to coupling with the handle assembly.

2. The introducer assembly as recited in claim 1, wherein the textured portion extends around an outer circumference of the sheath.

3. The introducer assembly as recited in claim 1, wherein the sheath is removable from instruments disposable therethrough.

4. The introducer assembly as recited in claim 1, further comprising a dilator disposed through the passage.

5. The introducer assembly as recited in claim 1, wherein the sheath is formed of PTFE (polytetrafluoroethylene).

6. The introducer assembly as recited in claim 1, wherein the sheath is mechanically bonded with the handle assembly.

7. The introducer assembly as recited in claim 1, wherein the interface includes one or more flow holes therein.

8. The introducer assembly as recited in claim 1, wherein the sheath includes molecularly oriented material, and the molecularly oriented material allows for the sheath to be removed from devices therein.

9. The introducer assembly as recited in claim 1, wherein the textured portion includes a chemically etched portion.

10. The introducer assembly as recited in claim 1, further comprising a valve sealingly associated with the sheath passage.

11. An introducer assembly comprising:
a sheath extending from a sheath proximal end to a sheath distal end, the sheath having a passage therethrough;
a handle assembly coupled with the sheath at an interface; and
the sheath is coupled with the handle assembly with a mechanical coupling and a chemically bonded coupling.

12. The introducer assembly as recited in claim 11, wherein the sheath is chemically etched at the interface.

13. The introducer assembly as recited in claim 11, wherein the sheath is splittable.

14. The introducer assembly as recited in claim 11, wherein the sheath is formed of molecularly oriented material.

15. The introducer assembly as recited in claim 11, wherein the mechanical coupling includes flow holes formed in the sheath.

16. The introducer assembly as recited in claim 11, where the sheath is formed of PTFE.

17. A method comprising:
forming a fluorinated polymer tube, the polymer tube forming a sheath extending from a sheath proximal end portion to a sheath distal end portion, the sheath having a passage therethrough;
texturizing the sheath proximal end portion and forming an interface; and
coupling a handle assembly with the sheath at the texturized interface.

18. The method as recited in claim 17, wherein texturizing includes chemically etching the interface.

19. The method as recited in claim 18, wherein chemically etching includes etching with sodium naphthalene/ethylene glycol dimethyl ether solution.

20. The method as recited in claim 17, wherein coupling the handle assembly with the sheath includes overmolding the handle assembly on to the sheath.

21. The method as recited in claim 17, wherein overmolding includes flowing material through holes of the sheath.

22. The method as recited in claim 17, wherein coupling the handle assembly includes mechanically and chemically coupling the sheath with the handle assembly.
